(19) United States
(12) Patent Application Publication
(10) Pub. No.: US 2005/0255595 A1
(43) Pub. Date: Nov. 17, 2005

(54) Genetically modified non-human mammal cells, procedure for their production and use in toxicity tests

(75) Inventors: Marco Tripodi, Trieste (IT); Maria Grazia Sacco, Trieste (IT); Laura Amicone, Trieste (IT); Paolo Maria Vezzoni, Trieste (IT)

Correspondence Address: GRAYBEAL, JACKSON, HALEY LLP, 155 - 108TH AVENUE NE, SUITE 350, BELLEVUE, WA, 98004-5901, US

(73) Assignee: CONSORZIO INTERUNIVERSITARIO PER LE BIOTECNOLOGIE

(21) Appl. No.: 11/056,941

(22) Filed: Feb. 11, 2005

(30) Foreign Application Priority Data
Aug. 14, 2002 (IT) TO2002A000729
Aug. 11, 2003 (WO) PCT/IT03/00506

(51) Int. Cl. C12Q 1/68; C12N 5/06; C12N 15/85

(52) U.S. Cl. 435/455; 435/354

(57) ABSTRACT

The invention concerns cells that can be used in toxicity tests and which therefore secrete, in response to toxic stress and stimuli, products that can be monitored and quantified. According to the present invention genetically modified cells are produced via the introduction of a first exogenous gene codifying a marker that can be activated in response to a pre-established external stimulus and a second exogenous gene that confers immortalisation: the first is preferably the human gene of the Growth Hormone and the second is preferably the human gene codifying a truncated form of the receptor of the Hepatocytic Growth Factor (Met gene). These cells perform hepatic functions and secrete into the culture medium the human growth hormone (GH) only in the case of toxicity or stress induced by organic or inorganic chemical or biological compounds. The fact that the GH secreted by MMH/GH cells is proportional, over a set interval, to the damage caused by the above-mentioned agents, permits use of the cells in toxicity tests in vitro.

### TECHNICAL FIELD

The present invention refers to genetically modified non-human mammal cells and, in particular, to bi-transgenic cells immortalised and differentiated, to use of the cells in cellular stress and toxicity tests induced by various organic and inorganic chemical compounds and to a procedure for production of the cells.

### BACKGROUND ART

Understanding of the molecular mechanisms of toxicity and the assessment of potential biological risks associated with the use of new substances forms a central part of the study and development of new therapeutic and industrial agents. The enormous number of new molecules developed annually requires the development of tests which are able to assess potential toxicity for humans associated with them and permit examination of the effects of new compounds also at the earliest stages of their development.

Generally, study of the effects of the compounds in question is based on analysis of survival or on biochemical tests correlated with toxicity.

It is known that for the study of toxicity, in vitro tests are widely used which employ primary hepatocytes in culture, since the hepatocyte is a cell that is particularly sensitive to toxic substances and therefore suited to said tests.

At the same time, the need is felt for improvements to the known techniques and, in particular, for availability of cells that permit analysis of the toxicity test results economically, quantitatively and rapidly.

Furthermore, the use of stabilised cells in toxicity tests is desirable; said cells, while maintaining the characteristics of the primary cells that can be obtained by organ explant, permit reduction of the extensive and enduring use of animals and also permit improved standardisation of the procedures.

### DISCLOSURE OF INVENTION

The aim of the present invention is therefore to provide cells that can be used in cellular toxicity and stress tests in vitro and the related production procedure.

According to the present invention a genetically modified mammal cell is provided, characterised in that it comprises a first exogenous gene codifying a marker that can be activated in response to an external stimulus and a second exogenous gene that confers immortalisation.

The cell is preferably a hepatocyte.

According to a preferred embodiment of the invention, the first gene is the human gene of the Growth Hormone and, in detail, consists of the sequence codifying the hGH placed under the transcriptional control of the Heat Shock Protein 70 (HSP70) promoter. According to a preferred embodiment of the invention, the second gene is the human gene codifying a truncated form of the Hepatocytic Growth Factor receptor and, in detail, consists of the entire transcriptional unit of the alpha-1-antripsin (AT) hepatospecific human gene in which the codifying sequence for the cytoplasmatic portion of the human receptor Met (tyrosine-kinase receptor of the Hepatocytic Growth Factor, cytoMET) is included, as the sole sequence codifying a proteinic product, within the second exon of the gene.

Preferably the cell is non-transformed, differentiated and polarised (i.e. it maintains a sectorial, cytoplasmatic and membrane specialisation, typical of the epithelial cell). In particular the hepatocytic cells according to the present invention are stable, immortalised, perform hepatic functions and secrete the human growth hormone following activation of the gene Hsp70 promoter, namely in case of toxicity or stress induced by organic or inorganic chemical or biological compounds.

The present invention also concerns a procedure for production of genetically modified mammal cells comprising the phase of inclusion of a first exogenous gene which codifies for a marker that can be activated in response to a pre-established external stimulus and a second exogenous gene that confers immortalisation.

Preferably the marker is a marker that can be induced by cellular stress and the cell is an immortalised hepatocyte, stable, non-differentiated and polarised.

Preferably the cells are obtained via genetic cross-breeding of transgenic animals, even more preferably said transgenic animals are mice or rats.

### BEST MODE FOR CARRYING OUT THE INVENTION

In detail, a preferred procedure for obtaining the cells according to the present invention is based on genetic cross-breeding between mice of a first transgenic family HSP-70/hGH and mice of a second transgenic family AT/cytoMET, which produces a bi-transgenic line consisting, in other words, of animals carrying both the transgenes, from which the liver is explanted.

Alternatively the cells are obtained by genic transmission, by transfection, by retroviral infection or by electroporation.

The cells of the present invention can be used for toxicity tests in vitro, preferably as biomarkers of ambient toxicity or as biomarkers of pharmacological toxicity.

According to the present invention, said cells can be used, preferably, in tests of toxicity induced by inorganic, organic non-cytotoxic and organic cytotoxic compounds. In particular, inorganic test compounds can be NaAsO2 and CdCl2, the non-cytotoxic organic compounds can be prostaglandins and analogues (2-cyclopentene-1-one and 1-octene-3-ol) thereof and the cytotoxic organic compounds can be BaP, TCHQ, CDNB and PCP.

According to the present invention it is also possible to produce a toxicological kit comprising the cells described previously.

The biological structure and activity of the single exogenous genes are known and are therefore not described in detail below, referring to the following publications, the content of which is incorporated herein by reference for the necessary parts:
1995-Amicone L et al Gene. 162, 323-328;
1997-Amicone L et al EMBO Journal 16, 495-503;
1997-Sacco M. G. et al Nature Biotech. 15: 1392-1397.

The cells of the present invention are subjected to growth and specific selection procedures (growth in medium with the addition of specific growth factors, bovine serum and antibiotics) permitting the derivation of cell lines hereinafter called MMH/GH.

Table 1 summarises the polarity markers and the hepatospecific genes expressed by the cells in point which highlight their similarity with the morphological-molecular behaviour of the hepatocyte in vivo.

TABLE 1

| Polarity | |
|---|---|
| Markers | Hepato-specific genes |
| E-Caderin | HNF1alpha |
| ZO-1 | HNF1beta |
| | HNF4 |
| | TTR |
| | Got1 |
| | Got2 |
| | LDH |
| | UDP-GT |
| | Cyp-1A1 |
| | Eph |

HNF: Hepatocyte Nuclear Factor; TTR: Transtiretine; Got: Glutamate oxaloacetate transaminase; LDH: Lactate dehydrogenase; UDP-GT: UDP glucuronosyl transferase; Cyp1A1::Cytochrome P450 1A1; Eph: Epxide hydrolase.

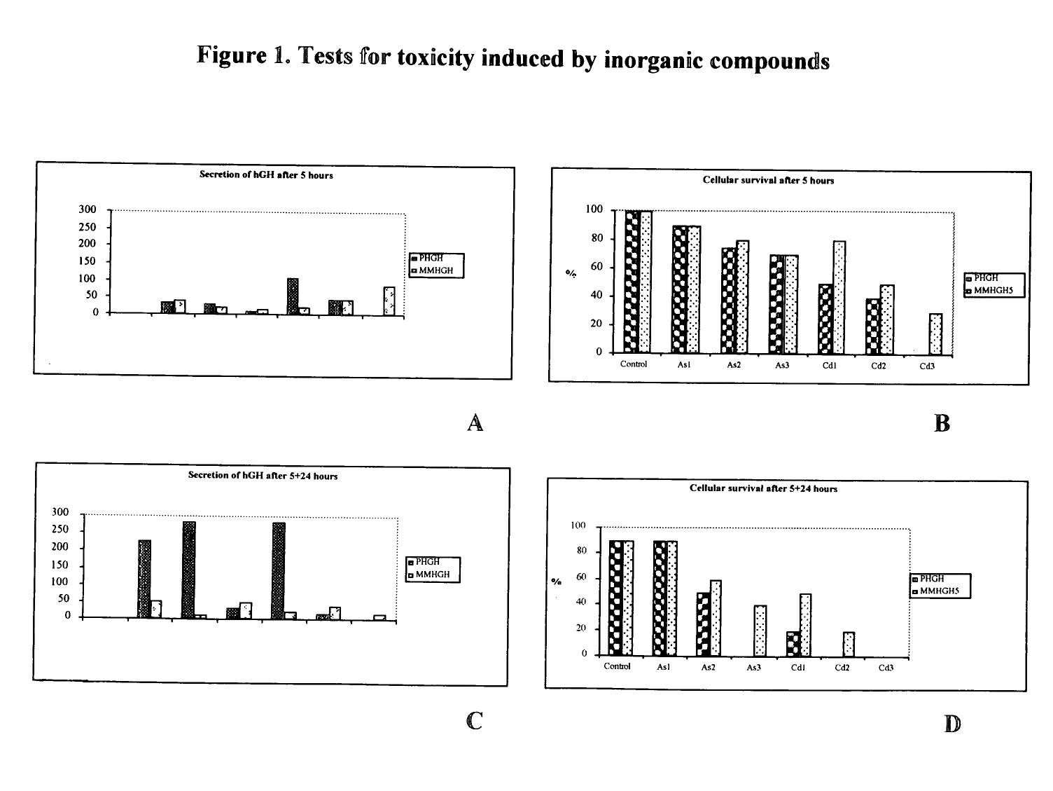

The invention will now be described on the basis of examples, without restricting it to said examples, and also with reference to the attached drawing which, in FIG. 1A,B,C,D illustrate graphs relating to a test of toxicity induced by inorganic compounds such as NaAsO2 and CdCl2, on a clone called MMH/GH5 of the cells of the present invention, comparing the results obtained with primary transgenic cultures (PHGH).

### EXAMPLE 1

Table 2 shows the results of a toxicity test performed with NaAsO2 and CdCl2 on 6 independent clones of cells obtained by genetically crossing mice of a transgenic family AT/cytoMET and mice HSP-70/hGH which resulted, due to liver explant performed in various phases of embryonal and post-natal development, in bi-transgenic lines, i.e. consisting of animals carrying both transgenes. These cells were called MMH/GHx, where x is an identification number of each clone.

The clones were tested for their ability to respond to toxic stimuli in vitro.

The basic levels of hGH secreted in the culture medium by cells MMH/GH at different stages and without any treatment (e.g. toxic or thermal) are below the sensitivity limit of the detection method.

The six clones were then treated with NaAsO2 and CdCl2 at the concentrations of 10⁻⁵, 5×10⁻⁵ and 10⁻⁴ M (called respectively As1, As2, As3, Cd1, CD2, Cd3) for 5 hours followed, if necessary, by 24 hours of recovery.

On the basis of the results illustrated, for the further analyses, clone no. 5 was selected, which was called MMH/GH5, since, as can be seen from table 2, it provides the best results. The response to the toxic stress of the clone MMH/GH5 was then compared with the one obtained in single transgenic HPS70/hGH primary hepatocytes (PHGH). A representative sample of the cell line derived from said clone MMH/GH5 was deposited in accordance with and for the purposes of the Treaty of Budapest on the international recognition of deposit of micro-organisms for the purposes of the patents procedure with the AID “Centro di Biotecnologie Avanzate (CBA), Interlab Cell Line Collection (ICLC)” Servizio Biotecnologie, Largo R. Benzi, 10, 16132 Genova, Italy, on Jul. 30, 2002, where it was given identification number PD 02007.

FIG. 1 shows the effects of the treatment of cells PHGH and MMH/GH5 with 10⁻⁵, 5×10⁻⁵ and 10⁻⁴ M of NaAsO2 and CdCl2 (As1, As2, As3 and Cd1, Cd2, Cd3 respectively) for 5 hours (graph A).

The responses obtained with CdCl2 have an inverse trend in the two types of cells in relation to concentration of the toxicant: the release of hGH in the culture medium of cells treated with CdCl2 is directly proportional to the concentration of the toxicant in the cells MMH/GH5 and is inversely proportional to it in the PHGHs. This different behaviour is explained by the greater sensitivity to the toxicant of the PHGH with respect to the MMH/GH5 as shown by analysis of the cellular vitality performed with the Trypan Blue method (FIG. 1, graph B). It should be noted, in fact, that survival of the PHGHs at the highest concentrations of the toxicant is practically nil. Therefore the greater resistance of the cells MMH/GH/5 to the toxicants constitutes another evident advantage as it permits the exclusion of false negatives in which the non-release of hGH, due to cellular death, is interpreted as non-toxicity of the compound. When the supernatant is collected after 5 hours of treatment followed by 24 hours of culture with normal medium (FIG. 1 graph C) the PHGHs respond, at the lowest concentrations of the two toxicants, with high levels of secretion of hGH and, at the highest concentrations, with low levels of secretion of hGH (lower cellular survival, graph D).

Contrary to the known behaviour of the PHGHs, the response of the MMH/GH5s can be measured in all the experimental conditions.

The results presented were obtained via six independent experiments in which each treatment was performed in triplicate.

TABLE 2

| | | 5 h | 5 + 24 h | | | 5 h | 5 + 24 h |
|---|---|---|---|---|---|---|---|
| Clone 2 | As1 | 0 | 0 | Clone 2 | Cd1 | 0 | 0 |
| | As2 | 55 | 40 | | Cd2 | 0 | 4 |
| | As3 | 0 | 0 | | Cd3 | 0 | 0 |
| | Basal | 0 | 0 | | Basal Activity | 0 | 0 |
| | Activity | | | | | | |
| Clone 4 | As1 | 0 | 0 | Clone 4 | Cd1 | 0 | 0 |
| | As2 | 0 | 0 | | Cd2 | 0 | 0 |
| | As3 | 0 | 0 | | Cd3 | 0 | 0 |
| | Basal Activity | 0 | 0 | | Basal Activity | 0 | 0 |
| Clone 5 | As1 | 42 | 45 | Clone 5 | Cd1 | 25 | 25 |
| | As2 | 30 | 15 | | Cd2 | 50 | 40 |
| | As3 | 20 | 45 | | Cd3 | 90 | 20 |
| | Basal Activity | 2 | 3 | | Basal Activity | 2 | 3 |
| Clone 9 | As1 | 70 | 60 | Clone 9 | Cd1 | 0 | 0 |
| | As2 | 110 | 0 | | Cd2 | 200 | 0 |
| | As3 | 0 | 0 | | Cd3 | 5 | 0 |
| | Basal Activity | 1 | 2 | | Basal Activity | 1 | 2 |
| Clone 10 | As1 | 15 | 15 | Clone 10 | Cd1 | 0 | 25 |
| | As2 | 7 | 4 | | Cd2 | 0 | 12 |
| | As3 | 0 | 7 | | Cd3 | 0 | 7 |
| | Basal Activity | 0 | 0 | | Basal Activity | 0 | 0 |
| Clone 11 | As1 | 0 | 0 | Clone 11 | Cd1 | 40 | 0 |
| | As2 | 0 | 0 | | Cd2 | 0 | 0 |
| | As3 | 25 | 0 | | Cd3 | 0 | 0 |
| | Basal Activity | 0 | 0 | | Basal Activity | 0 | 0 |

### EXAMPLE 2

Table 3 describes the performance of toxicity tests on MMH/GH cells with non-cytotoxic organic compounds, such as prostaglandins and prostaglandin analogues, and comparison with PHGH, as in the previous example.

In table 3 the cellular survival percentage was determined with the Trypan Blue exclusion method. The values are expressed as pg of hGH secreted in the supernatant every 10⁶ cells.

For treatment, the two types of cells were treated with different concentrations of the compounds for 1 or 5 hours and then with normal medium for a further 24 hours. The effects of the treatment were assessed as described previously for the inorganic toxicants.

The levels of hHG increase in the medium when treatment with the substances is followed by 24 hours of culture in normal medium which permits the accumulation of hGH. The treatment with 2-cyclo-pentene- 1-one(2 Cycl), a synthetic analogue of the prostaglandins described as stimulator of the promoter HSP70, induces a rapid response in the MMH/GH5s and the highest secretion levels are observed after 24 hours of growth in normal medium.

TABLE 3

| | | | | Primary | | | | | | | | | |
|---|---|---|---|---|---|---|---|---|---|---|---|---|---|
| | | | | hepatocytes | | | | | | | | | |
| | | | | (PHGH) | | | | | | MMHGH/5 | | | |
| | | 1 h | 5 h | 1 + 24 h | % | 5 + 24 | % | 1 h | 5 h | 1 + 24 h | % | 5 + 24 | % |
| | Control | 1 | 1 | 0 | 95 | 0 | 95 | 0 | 0 | 0 | 95 | 0 | 90 |
| PGA1 | 25 mM | 9 | 16 | 13 | 95 | 69 | 90 | 10 | 27 | 0 | 90 | 2 | 90 |
| PGA1 | 50 mM | 7 | 17 | 4 | 90 | 522 | 70 | 94 | 47 | 116 | 90 | 9 | 90 |
| PGA2 | 25 mM | 1 | 56 | 0 | 90 | 10 | 90 | 1 | 53 | 2 | 90 | 0 | 90 |
| PGA2 | 50 mM | 27 | 95 | 26 | 90 | 37 | 90 | 8 | 107 | 92 | 90 | 0 | 90 |
| PGD | 25 mM | 6 | 30 | 5 | 90 | 2 | 90 | 0 | 7 | 0 | 90 | 0 | 90 |
| PGD | 50 mM | 78 | 38 | 27 | 90 | 5 | 90 | 0 | 4 | 0 | 90 | 0 | 90 |
| PGE | 25 mM | 3 | 66 | 257 | 90 | 32 | 90 | 0 | 2 | 0 | 90 | 0 | 90 |
| PGE | 50 mM | 2 | 70 | 41 | 90 | 12 | 90 | 0 | 0 | 0 | 90 | 0 | 90 |
| 1-Oct | 0.5 mM | 1 | 16 | 51 | 90 | 15 | 60 | 4 | 4 | 3 | 90 | 0 | 50 |
| 1-Oct | 1 mM | 2 | 6 | 20 | 50 | 69 | 20 | 7 | 10 | 0 | 70 | 0 | 20 |
| 2-Cycl | 0.5 mM | 7 | 33 | 103 | 90 | 126 | 90 | 104 | 8 | 0 | 90 | 0 | 90 |
| 2-Cycl | 1 mM | 7 | 12 | 159 | 80 | 44 | 60 | 65 | 18 | 0 | 90 | 3 | 90 |

The effect of the treatment with 1-octene-3-ol (1 Oct), another analogue of the prostaglandins not activating the promoter HSP70, can be measured only in PHGH at the end of the treatment and is associated with a reduced cellular survival. As expected from the fact that the prostaglandins perform a physiological role, the vitality of both the PHGHs and the MMHGH cells is high after treatment with these substances.

Since the activation of heat shock (HS) genes and the cytoprotective role performed by HSP70 have been described in many human pathologies, our model could be used to identify new activators of the promoter HSP70 for use in therapeutic protocols aimed at protecting human cells in a condition of stress via an increase in the production of hsp.

### EXAMPLE 3

Table 4 concerns toxicity tests on MMH/GH cells with cytotoxic organic compounds and comparison with PHGH. Response to the stress mediated by the HS proteins was widely studied using heavy metals and heat as inductors. Recently the use of these proteins as biomarkers has been proposed in the monitoring of ambient pollution. The effects of the ambient organic pollutants were assessed in our model. The PHGHs secrete hGH only after treatment with said substances is followed by 24 hours of culture in a normal medium. This behaviour can be explained by a slower absorption of the organic compounds with respect to the inorganic compounds or by the need for chemical modifications that must take place in the cell.

TABLE 4

| | | PHGH | | | | | | MMH/GH | | | | | |
|---|---|---|---|---|---|---|---|---|---|---|---|---|---|
| | | 1 h | 5 h | 1 + 24 h | % | 5 + 24 h | % | 1 h | 5 h | 1 + 24 h | % | 5 + 24 h | % |
| | Control | 0 | 0 | 2 | 95 | 2 | 90 | 0 | 1 | 0 | 95 | 2 | 90 |
| BaP | 10 mM | 0 | 0 | 0 | 50 | 0 | 50 | 3 | 0 | 21 | 90 | 116 | 90 |
| BaP | 50 mM | 0 | 23 | 0 | 50 | 0 | 30 | 0 | 0 | 92 | 90 | 149 | 90 |
| PCP | 50 mM | 0 | 0 | 0 | 10 | 29 | 10 | 0 | 51 | 7 | 90 | 78 | 70 |
| PCP | 100 mM | 0 | 0 | 0 | 0 | 16 | 0 | 0 | 0 | 14 | 80 | 87 | 50 |
| TCHQ | 50 mM | 0 | 0 | 190 | 50 | 270 | 60 | 0 | 0 | 121 | 50 | 129 | 30 |
| TCHQ | 100 mM | 0 | 0 | 109 | 0 | 131 | 0 | 10 | 0 | 97 | 10 | 119 | 0 |
| CDNB | 5 mM | 0 | 7 | 34 | 80 | 113 | 40 | 27 | 115 | 65 | 80 | 79 | 60 |
| CDNB | 50 mM | 0 | 0 | 0 | 5 | 26 | 5 | 74 | 46 | 191 | 70 | 58 | 20 |

BaP = Benzo(a)Pyrene;
PCP = PentaChloroPhenol;
TCHQ = TetraChloroHydroQuinone;
CDNB = 1-Chloro-2,4-DiNitro-Benzene
% = vitality of the cells determined by an exclusion test with Trypan Blue
The values are expressed as picograms of hGH released in the medium by 10⁶ cells

As illustrated in Table 4, the benzo-a-pyrene (BaP) does not stimulate the promoter HSP70 in the PHGHs while in the MMH/GH5s a dosable response is obtained when the treatment is followed by 24 hours of culture in normal medium.

The penta-chloro-phenol (PCP), on the other hand, which is toxic for the PHGHs, requires a prolonged exposure time to induce a response in MMH/GH5.

The TetraChloroHydroQuinone (TCHQ), which is the most reactive metabolite of the PCP in vivo when metabolised by the cytochrome P450, is a known oxidising agent and produces a stronger response in both the cellular models.

The 1-Chloro-2,4-DiNitro-Benzene (CDNB) is a known purifier of the reduced glutathione (GSH). This compound induces a considerable secretion of the hGH from the MMH/GH5 cells in all the conditions tested and proves to be less toxic on these cells than on the PHGHs, as demonstrated by the data in table 4.

1. Genetically modified mammal cell, characterised in that it comprises a first exogenous gene codifying a marker that can be activated in response to a pre-established external stimulus and a second exogenous gene that confers immortalisation.

2. Cell according to claim 1, characterised in that said marker can be induced by cellular stress.

3. Cell according to claim 2, characterised in that it is a hepatocyte.

4. Cell according to claim 3, characterised in that said first gene is the human gene of the Growth Hormone.

5. Cell according to claim 3, characterised in that said second gene is the human gene codifying a truncated form of the receptor of the Hepatocytic Growth Factor (MET gene).

6. Cell according to claim 5, characterised in that the second gene consists of the entire transcriptional unit of the alpha-1-antripsin (AT) hepatospecific human gene in which the codifying sequence for the cytoplasmatic portion of the tyrosine-kinase receptor of the Met human Hepatocytic Growth Factor (cytoMET) is included, as the sole sequence codifying a proteinic product, within the second exon of the gene.

7. Cell according to claim 6, characterised in that said first gene consists of the sequence codifying the hGH placed under the transcriptional control of the Heat Shock Protein 70 (HSP70) promoter.

8. Cell according to claim 1, characterised in that it is of murine origin.

9. Cell according to claim 1, characterised in that it is non-transformed and differentiated.

10. Cell according claim 1, characterised in that it is polarised.

11. Procedure for the production of genetically modified mammal cells, characterised in that it comprises the phase of inclusion of a first exogenous gene that codifies for a marker that can be activated in response to a pre-established external stimulus and a second exogenous gene that confers immortalisation.

12. Procedure according to claim 11, characterised in that said marker can be induced by cellular stress.

13. Procedure according to claim 12, characterised in that said mammal cell is a murine hepatocyte.

14. Procedure according to claim 11, characterised in that the first gene is the human gene of the Growth Hormone.

15. Procedure according to claim 14, characterised in that the second gene is the human gene codifying a truncated form of the receptor of the Hepatocytic Growth Factor.

16. Procedure according to claim 11, characterised in that the second gene consists of the entire transcriptional unit of the alpha-1-antripsin (AT) hepatospecific human gene in which the codifying sequence for the cytoplasmatic portion of the Met human receptor (cytoMET) is included, as the sole sequence codifying a proteinic product, within the second exon of the gene.

17. Procedure according to claim 16 characterised in that the first gene consists of the sequence codifying the hGH placed under the transcriptional control of the Heat Shock Protein 70 (HSP70) promoter.

18. Procedure according to claim 11, characterised in that said hepatocytic cells are obtained by means of liver explant in the products of genetic cross-breeding of transgenic animals.

19. Procedure according to claim 18, characterised in that the transgenic animals are mice or rats.

20. Procedure according to claim 19, characterised in that said cells are obtained by cross-breeding mice of a transgenic family AT/cytoMET with mice of a transgenic family HSP-70/hGH.

21. Procedure according to claim 11, characterised in that said cells are obtained by genic transmission.

22. Procedure according to claim 11, characterised in that said cells are obtained by transfection.

23. Procedure according to claim 11, characterised in that said cells are obtained by retroviral infection.

24. Procedure according to claim 11, characterised in that said cells are obtained by electroporation.

25. Use of cells according to claim 1, for in vitro toxicity tests.

26. Use according to claim 25 characterised in that said test is a test for toxicity induced by inorganic compounds.

27. Use according to claim 25, characterised in that said inorganic compounds are NaAsO2 or CdCl2.

28. Use according to claim 25, characterised in that said test is a test for toxicity induced by non-cytotoxic organic compounds.

29. Use according to claim 28, characterised in that said non-cytotoxic organic compounds are prostaglandins and analogues thereof.

30. Use according to claim 25, characterised in that said test is a test for toxicity induced by cytotoxic organic compounds.

31. Use according to claim 30, characterised in that said cytotoxic organic compounds are selected from the group consisting of BaP, TCHQ, CDNB and PCP.

32. Use of cells according to claim 1, as biomarkers of ambient toxicity.

33. Use of cells according to claim 1, as biomarkers of pharmacological toxicity.

34. Toxicological kit, characterised in that it uses hepatocytic cells according to claim 1.

35. Murine hepatocyte cellular line characterised in that it expresses the growth hormone hGH in response to a pre-established external stimulus, a representative sample of which was deposited with the CBA-ICLC (Centro di Biotecnologie Avanzate—Interlab Cell Line Collection) of Genova under number PD 02007 on Jul. 30, 2002.
